(19) United States
(12) Patent Application Publication
(10) Pub. No.: US 2026/0102595 A1
(43) Pub. Date: Apr. 16, 2026

(54) ORAL DRUG DELIVERY DEVICE

(71) Applicant: Biograil APS, Roskilde (DK)

(72) Inventors: Nikolaj Skak, Roskilde (DK); Karsten Lindhardt, Roskilde (DK); Martin Rex Olsen, Roskilde (DK); Henrik Bang Mikkelsen, Roskilde (DK)

(21) Appl. No.: 19/114,980

(22) PCT Filed: Oct. 27, 2023
(86) PCT No.: PCT/EP2023/080160

§ 371 Date: Mar. 25, 2025

(30) Foreign Application Priority Data
Oct. 28, 2022 (DK) PA202270525

(51) Int. Cl. A61M 31/00

(52) U.S. Cl. CPC A61M 31/002; A61M 2210/1053; A61M 2210/106; A61M 2210/1064

(57) ABSTRACT

An oral drug delivery device (100) comprising a housing (102) having a longitudinal axis (104) and extending from a first end to a second end, a first attachment part (110) having a first distal end (112) and a first proximal end (114), the first attachment part connected within the housing and configured to deliver an active drug substance (154), and an actuator (116) engaged with the first attachment part and configured to rotate the first attachment part about a first rotation axis (118), wherein the first rotation axis optionally forms an angle larger than 45 degrees with the longitudinal axis.

The present disclosure relates to a drug delivery device and in particular to a drug delivery device for oral administration. The drug delivery device is advantageously configured for delivery of an active drug substance in the gastrointestinal tract including the stomach and/or intestines, such as the small intestines and/or the large intestines (colon).

### BACKGROUND

A number of low permeable and/or low water soluble active drug substances are currently delivered by i.e. subcutaneous, intradermal, intramuscular, rectal, vaginal or intravenous route. Oral administration has the potential for the widest patient acceptance and thus attempts to deliver low permeable and/or low water soluble active drug substances through the preferred oral route of administration has been tried but with limited success in particular due to lack of stability and limited absorption from the gastrointestinal tract.

Stability both relates to the stability of the active drug substance during manufacturing and storage of the delivery device, and to the stability of the active drug substance during the passage in the gastrointestinal tract before it become available for absorption.

Limited gastrointestinal absorption is due to the gastrointestinal wall barrier preventing active drug substance from being absorbed after oral dosing because of the low permeability of the active drug substance, which is for example due to pre-systemic metabolism, size and/or the charges and/or because of the water solubility of the active drug substance.

Multiple approaches to solve these stability and absorption challenges have been suggested, but an effective solution to the challenges remain unresolved.

### SUMMARY

Thus, there is an unmet need to provide a drug delivery device, which is capable of delivering drug substances for absorption in the gastrointestinal tissue. More generally, there remains a need for drug products and methods that enable enhanced drug delivery, when drug products are administered orally to patients.

Disclosed herein is an oral drug delivery device. The oral drug delivery device comprises a housing having a longitudinal axis and extending from a first end to a second end. The oral drug delivery device comprises a first attachment part having a first distal end and a first proximal end, the first attachment part connected to and/or within the housing and configured to deliver an active drug substance. The oral drug delivery device comprises an actuator engaged with the first attachment part and configured to rotate the first attachment part about a first rotation axis, e.g. in relation to the housing. The first rotation axis optionally forms an angle larger than 45 degrees with the longitudinal axis.

It is an advantage of the oral drug delivery device that the oral drug delivery device, such as via the first attachment part, can efficiently attach to a patient's system, such as for delivering an active drug substance to the patient. The oral drug delivery device can include minimum moving components, thereby reducing the amount of parts that could break and/or fail during use. Further, certain implementations of the disclosed oral drug delivery device do not rely on multiple attachment parts, thereby improving the attachment ability of the oral drug delivery device to the patient, such as gastrointestinal tissue, e.g. in the stomach and/or intestines.

Advantageously, the present disclosure provides for a large difference in inertia between the housing/body and the attachment part(s) thereby causing only or main movement of the attachment part during insertion in turn providing efficient insertion of the attachment part(s) in the tissue.

Further, increased design flexibility of the oral drug delivery device is provided, e.g. by enabling higher flexibility in spike tip/distal end insertion angle in the tissue and/or larger reach of the spike by providing a larger spike tip to rotational axis center distance.

Other advantages of the present disclosure include simplicity in design, large spike, miniaturization, small space footprint in turn allowing for more volume and flexibility of high load device.

### BRIEF DESCRIPTION OF THE DRAWINGS

The above and other features and advantages of the present invention will become readily apparent to those skilled in the art by the following detailed description of example embodiments thereof with reference to the attached drawings, in which:

FIG. 1 shows an exploded view of an example oral drug delivery device,

FIGS. 2-6 show different views of the example oral drug delivery device of FIG. 1,

FIG. 7 shows an exploded view of an example oral drug delivery device,

FIGS. 8-12 show different views of the example oral drug delivery device of FIG. 7,

FIG. 13 shows an exploded view of an example oral drug delivery device,

FIGS. 14-20 show different views of the example oral drug delivery device of FIG. 13,

FIGS. 21-26 show different views of an example oral drug delivery device,

FIGS. 27-30 show different views of an example oral drug delivery device,

FIGS. 31-32 show different views of an example oral drug delivery device, and

FIGS. 33-34 show different views of an example oral drug delivery device.

### DETAILED DESCRIPTION

Various example embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments and the functionalities associated therewith. They are not intended as an exhaustive description of the invention or as a limitation on the scope of the invention or the physical appearance of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

Disclosed herein is an oral drug delivery device. In other words, a drug delivery device configured for oral administration is provided. The oral drug delivery device comprises a housing or body having a longitudinal axis and extending from a first end to a second end.

The drug delivery device optionally comprises a shell or cover, e.g. encapsulating the other parts of the drug delivery device. The shell may be made of a biodegradable material, such as gelatine and/or Hydroxypropyl methyl cellulose (HPMC). Other suitable materials for the shell includes compositions comprising one or more polymethacrylate-based copolymers also known as Eudragits, e.g. Eudragit E PO. The shell may be a multipart shell, such as a two-part capsule. The shell or capsule may comprise a plurality of layers, e.g. a first layer of a first material and a second layer of a second material different from the first material.

The oral drug delivery device comprises one or more, such as two, attachment parts including a first attachment part having a first distal end and a first proximal end. The first attachment part is connected to and/or within the housing and configured to deliver an active drug substance, e.g. in an attached position.

The first attachment part, such as the first spike described later, may be made of or comprise a biodegradable material. For example, the first attachment part, such as the first spike, may be made of a material comprising one or more starch-based polymers, one or more cellulose-based polymers, one or more synthetic polymers, or a combination thereof, such as mixtures of starch-based and synthetic polymers or mixtures of starch-based and cellulose-based polymers.

In one or more examples, the first attachment part, such as the first spike, comprises one or more biodegradable polymers selected from polylactic acid (PLA), one or more derivatives of PLA, polycaprolactone (PCL), one or more derivatives of PCL, polyhydroxybutyrate (PHB), one or more derivatives of PHB.

In one or more examples, the first attachment part, such as the first spike, comprises or consist of PLA, ethyl cellulose, one or more plasticizers (such as any of the plasticizers described herein) and one or more UV stabilisers (such as any of the UV stabilisers described herein), optionally including an active drug substance.

In one or more examples, the material of the first attachment device, may comprise a metal or metal alloy, such as a biodegradable or bioresorbable metal or metal alloy, e.g. magnesium, zinc, and/or iron or an alloy comprising one or more of magnesium, zinc and iron. A biodegradable or bioresorbable metal or metal alloy may be understood as a metal or metal alloy that degrades safely within a body, such as a human body, in a practical amount of time, for example related to their application. A metallic first attachment part may be preferred in examples where the first attachment part, such as the first spike, have or provides access to a first chamber also denoted drug cavity, e.g. to hold, such as retain, an active drug substance.

In one or more examples, the first attachment part, such as the first spike, is made of a material comprising or being a biodegradeable polymer as described above, the first attachment part, such as the first spike, comprising a drug cavity with, holding, or accommodating an active drug substance.

In one or more examples, the first attachment part, such as the first spike, is made of a material comprising a biodegradeable and/or bioresorbable polymer, e.g. where active drug substance is embedded in or dispersed in the polymer, such as polyethylene glycol (PEG), PEG derivatives, polyethylene oxide (PEO), PEO derivatives, or combinations thereof.

In one or more examples, the first attachment part, such as the first spike, is made of a material comprising one or more co-polymers, such as one or more poloxamers. The one or more poloxamers may include Poloxamer 188 and/or Poloxamer 407.

The oral drug delivery device comprises an actuator engaged with the first attachment part and/or the housing and configured to rotate the first attachment part about a first rotation axis in relation to the housing. In one or more example oral drug delivery devices, the first rotation axis forms an angle larger than 45 degrees with the longitudinal axis. In one or more example oral drug delivery device, the first rotation axis forms an angle larger than 75 degrees, such as in the range from 80 to 100 degrees, with the longitudinal axis.

In one or more examples, an oral drug delivery device is disclosed, the oral drug delivery device comprising a housing having a longitudinal axis and extending from a first end to a second end; a first attachment part having a first distal end and a first proximal end, the first attachment part connected to and/or within the housing and configured to deliver an active drug substance; and an actuator engaged with the first attachment part and configured to rotate the first attachment part about a first rotation axis, wherein the first rotation axis forms an angle larger than 45 degrees with the longitudinal axis.

The oral drug delivery device may have an initial configuration. The initial configuration may be an initial position and/or an initial state, such as when the oral drug delivery device is taken into a patient, such as via swallowing. The initial configuration can be a configuration before any actuation of an actuator, e.g., an actuator mechanism, of the oral drug delivery device. The initial configuration can be when the attachment part(s), e.g., the first attachment part and/or the second attachment part, are in their initial position.

The oral drug delivery device may have a size and geometry designed to fit into a pharmaceutical composition for oral administration. As used herein, drug delivery device refers to an oral drug delivery device unless otherwise stated.

The oral drug delivery device may be configured to be taken into the body via the oral orifice. For example, the oral drug delivery device may be configured to be taken into the body in an initial configuration, e.g., when the first attachment part and/or the second attachment part is in their respective initial position. Thus, the outer dimensions of the oral drug delivery device may be small enough for a user to swallow. The oral drug delivery device may be adapted to carry a drug substance, e.g., an active drug substance, into the body of the user, via the digestive system, so that the drug delivery device may e.g., travel from the mouth of the user into the stomach, via the oesophagus. The oral drug delivery device may be configured to further travel into the intestines from the stomach, and may optionally travel into the bowels and out through the rectum. The drug delivery device, such as the housing, may have a length in the range from 10 mm to 30 mm. The housing may be a single-part or multi-part housing.

The oral drug delivery device may be configured to deliver the drug in any part of the digestive system of the user, where in one example it may be configured to deliver a drug substance into the stomach of the user. In another example, the oral drug delivery device may be adapted to initiate the drug delivery when the device has passed the stomach and has entered the intestine of the user. In other words, the oral drug delivery device may be configured to attach to a wall of the stomach or a wall of the intestines, e.g., depending on the desired release position of the active drug substance. For example, the oral drug delivery device may be configured to transition from the initial configuration to an attached configuration to attach to the patient. In one or more example oral drug delivery devices, the oral drug delivery device is configured to transition from the initial configuration to the attached configuration as the first attachment part and/or the second attachment part transitions from the respective initial position to attached position.

The attachment part(s) of the oral drug delivery device, e.g., the first attachment part and/or the second attachment part, may be configured to interact, such as penetrate, with the inner surface linings of the gastrointestinal tract, so that the oral drug delivery device may e.g., be attached to the inner surface (mucous membrane) of the stomach, or alternatively to the mucous membrane of the intestines, such as in the attached position. The attachment part(s), e.g. first attachment part and/or second attachment part, may be configured to interact with the mucous membranes, e.g., in order to fix or attach the oral drug delivery device, e.g., for a period of time, inside the body of the user. By attaching the oral drug delivery device, it will allow a drug substance to be delivered into a part of the digestive system, in order to provide a drug substance to the body of the user. The attachment part(s) may be configured to interact with, such as penetrate, the mucous membranes, e.g., in order to inject drug substance into the gastrointestinal tract wall.

In one or more example drug delivery devices, the oral drug delivery device may be configured with detachment capabilities. For example, the oral drug delivery device may be constructed in a way that releases the oral drug delivery device or at least parts thereof from internal tissues and/or internal surfaces, such as after distribution of the active drug substance.

The oral drug delivery device may have an attached configuration, e.g., attached position, attachment position, attachment configuration, attached state. The attached configuration may allow for the oral drug delivery device to attach to a surface in the patient, such as tissue, which can allow for delivery of the active drug substance. The attached configuration can be a configuration for attachment of the drug delivery device to the patient. The attached configuration may be different from the initial configuration. In the attached configuration, the oral drug delivery device may remain attached to the patient. In the attached configuration, the first attachment element and/or the second attachment element is in the attached position. The attached configuration can be a configuration after an actuation of the actuator of the oral drug delivery device.

In one or more example oral drug delivery devices, the oral drug delivery device can be defined by a housing. The housing includes a first end and a second end, which can be considered a first longitudinal end and a second longitudinal end. The first end is opposite the second end. The housing includes a longitudinal axis. The longitudinal axis extends between the first end and the second end. In one or more oral drug delivery devices, the housing can be configured to retain certain components of the oral drug delivery device in an initial position. For example, the housing can retain one or more of the first attachment part and the actuator. The housing can retain a second attachment part. The housing can form a first cavity and/or a second cavity as discussed herein. The housing can be configured to at least partially contain (e.g., surround) components of the oral drug delivery device. For example, the housing can at least partially contain the actuator. The housing can at least partially contain the first attachment part.

In one or more oral drug delivery devices, the housing may be a one-part or single-part body part, e.g., unitary. In one or more oral drug delivery devices, the housing or body can be made up of one or more body parts, such as first body part and a second body part. The housing includes an outer surface. In one or more oral drug delivery devices, the housing may be non-rotational. For example, while internal components of the oral drug delivery device may rotate with respect to the housing while the housing remains stationary (outside of inertial forces). In other words, if the housing is formed from two body parts attached to one another, such as a first body part and a second body part, the first body part may be non-rotational with the second body part. For example, the first body part is non-rotationally fixed to the second body part.

In one or more example oral drug delivery devices, the housing may define a first cavity, such as a recess, gap, slot, hole, aperture, retainer, first housing cavity, extending to an outer surface of the housing. The first cavity may be formed by solid walls on at least two sides except outermost surface(s), which is/are open. For example, the first cavity may be formed by a first wall, a second wall, and optionally a bottom. The first wall may be opposite the second wall. The bottom may connect the first wall and the second wall. In one or more example oral drug delivery devices, the first wall and/or the bottom wall and/or the second wall are flat. In one or more example oral drug delivery devices, the first wall and/or the bottom wall and/or the second wall are curved. The open outermost surface may be curved to follow along with an outer surface of the oral drug delivery device. The housing may include a plurality of cavities, such as a second cavity, third cavity, fourth cavity, etc. For example, a first cavity may be located on an opposite side of the housing, such as an opposite side of the longitudinal axis, from a second cavity and/or a third cavity. Components of the oral drug delivery device within the first cavity may be considered to be contained within the housing.

The oral drug delivery part may include one or more attachment part(s), e.g., a first attachment part and/or a second attachment, of the oral drug delivery device. The one or more attachment part(s) may be seen as any kind of attachment part(s) that may be capable of attaching the oral drug delivery device to a biological tissue, such as a stomach wall, a wall of the bowels and/or intestines of a human or animal body. The attachment part(s), e.g., first attachment part and/or second attachment, may be configured to attach the drug delivery device to a patient. The attachment part(s), e.g., first attachment part and/or a second attachment, may be configured to deliver an active drug substance. The oral drug delivery part may have only a single attachment part.

The first attachment part can be connected within the housing. The first attachment part may be translatably attached with respect to the housing. The first attachment part may be configured to move with respect to the housing. For example, the first attachment part may be rotationally attached within the housing, such as via the first proximal end of the first attachment part. The first attachment part may be rotationally connected to and/or within the first cavity. The first attachment part can be configured, via the actuator, to transition from an initial position to an attached position. In the initial position, the first attachment part or at least parts or sections thereof may be located within the first cavity. For example, a first distal end and/or a distal section of the first attachment part may be located within the first cavity in the initial position. The first distal end can exit or “clear” the first cavity and/or the housing when transitioning from the initial position to the attached position. To clear the first cavity can include being outside of the housing, such as outside of the first cavity.

The first attachment part may comprise a single component, such as being unitary. The first attachment part may comprise multiple components. In one or more example oral drug delivery devices, the first attachment part comprises a first arm. In one or more example oral drug delivery devices, the first attachment part comprises a first spike. The first spike can be arranged at the first distal end. The first attachment part, such as the first spike, can include a bend.

In other words, the first attachment part may comprise a first arm and/or a first spike, e.g. spike component, needle, spike, spear. The first attachment part has a first proximal end, e.g. proximal end, and a first distal end, e.g. distal end. A first tip of the first spike can form the first distal end. In other words, the first distal end is a first tip of the first spike. The first arm may be arranged at, form a part of, or constitute the first proximal end of the first attachment part. The first arm may be connected to and/or within the housing, such as at the first proximal end of the first attachment part.

The first spike may have a length in the range from 1 mm to 15 mm such, as in the range from 3 mm to 10 mm. Thereby sufficient penetration into the internal tissue may be provided for while at the same time reducing the risk of damaging the internal tissue. The first distal end of the first attachment part may be provided with a tip configured to penetrate a biological tissue. The first distal end of the first attachment part may be provided with a gripping part configured to grip a biological tissue.

The first spike may be straight and/or curved. The first attachment part may include one, two, or more straight portions formed at an angle, thereby forming the bend therebetween.

The bend may be in the range from 30 degrees to 120 degrees, such as in the range from 75 degrees to 105 degrees, e.g. 90 degrees. In one or more example oral drug delivery devices, the first spike comprises a proximal section. In one or more example oral drug delivery devices, the first spike comprises a distal section including the distal end. The distal end points in a distal direction being parallel to the distal section. In one or more example oral drug delivery devices, the distal section extends at an angle between 30-80 degrees or at an angle between 100-170 degrees from the longitudinal axis in an attached position. In other words, the distal direction may, in the attached position, form an angle in the range from 30 degrees to 80 degrees with the longitudinal axis. In one or more example oral drug delivery devices, the distal section extends at an angle between 30-80 degrees from the longitudinal axis in an attached position. In one or more example oral drug delivery devices, the distal section extends at an angle between 100-170 degrees from the longitudinal axis in an attached position. In other words, the distal direction may, in the attached position, form an angle in the range from 100 degrees to 170 degrees with the longitudinal axis. The angle between the distal section and the proximal section can be formed by a curve. The angle between the distal section and the proximal section can be formed by a single change of angle. The bend can separate the proximal section from the distal section, such as connecting the proximal section to the distal section. The distal section can extend at an angle between 30-80 degrees or at an angle between 100-170 degrees from the longitudinal axis when the distal end has cleared the housing, such as cleared the first cavity. The angle can be formed in a plane of rotation of the first attachment part. The angle can be formed at a second angle from the plane of rotation of the first attachment part.

In one or more example oral drug delivery devices, the first attachment part may include one or more components, e.g. parts, intermediates, pieces. One or more of the one or more components may be releasably connected to the oral drug delivery device, such as the housing.

The first attachment part, such as the distal section, can be held within the first cavity of the housing, e.g. in the initial position. In one or more example oral drug delivery devices, the first attachment part can be translatable connected to the housing. The first attachment part can be rotatably connected to the housing. In an initial configuration, the first attachment part or parts thereof, such as the distal section, can be held fully within the first cavity. This can be known as the initial position. In an attached configuration, the first attachment part can may at least partially extend out of the first cavity. Thus, a distal end of the first attachment part can translate, such as rotate, from within the first cavity, such as in the initial position, to outside of the first cavity, such as the attached position. This movement can occur during operation of the actuator.

While rotational motion is discussed herein, it will be understood that other translations and/or motions can be used as well, e.g., as an alternative or in combination with rotational motion. For example, straight, longitudinal, radial, and circumferential motion can all be used to translate the first attachment part between the initial position and the attached position. Further, as used herein translate can include one or more of shifting, rotation, moving, and releasing.

In one or more example oral drug delivery devices, a proximal end of the first attachment part may include a first attachment point (e.g., attachment surface, attachment mechanism, attachment plane, attachment section, attachment component, attachment structure, attachment, connection, rotatable connection) within the housing. For example, the first attachment part may include a first attachment point within the housing. The first attachment point may be, for example a hinge. The first attachment point may be a pin. The type of attachment point is not limiting. The first attachment part may comprise a first hinge part, e.g. arranged at the proximal end of the first attachment part. The first hinge part may comprise a bore, such as a through-going bore, e.g. configured to accommodate a second hinge part or rod for forming a rotatable connection between the housing and the first attachment part.

Accordingly, the housing may comprise a second hinge part, such as a rod. The actuator, such as a spring, may be arranged between the first hinge part and the second hinge part, e.g. circumferentially to the second hinge part and/or at least partly in the through-going bore.

In one or more example oral drug delivery devices, the first attachment part can be configured to translate at the first attachment point with respect to the housing. In one or more example oral drug delivery devices, the first attachment part can be configured to rotate at the first attachment point with respect to the housing. The rotation of the first attachment part can be about a first rotation axis. For example, the first attachment part may be rotationally connected within the first cavity, e.g., with respect to the housing, along a first rotation axis.

The first rotation axis may be different from the longitudinal axis. For example, the first rotation axis can form an angle larger than 45 degrees with the longitudinal axis. The first rotation axis can form an angle larger than 75 degrees with the longitudinal axis. In the present context, an angle between two axes can be seen as the smallest angle between the two axes. For example, if there is a primary angle and a secondary angle, the smallest angle is the primary angle.

In one or more example oral drug delivery devices, the first rotation axis is orthogonal to the longitudinal axis. For example, the first rotation axis can form an angle of 90 degrees with the longitudinal axis. In one or more example oral drug delivery devices, the first rotation axis forms an angle less than 15 degrees with the longitudinal axis. In one or more example oral drug delivery devices, the first attachment part can be configured to rotate along a plane formed by the longitudinal axis. In one or more example oral drug delivery devices, the first attachment part can be configured to rotate along a single plane formed by the longitudinal axis.

In one or more example oral drug delivery devices, the first rotation axis is stationary. In particular, the first rotation axis is stationary with respect to the housing. Accordingly, while the oral drug delivery may rotate in space, the first rotation axis is always positioned at the same relative position to the housing. For example, inertia may rotate the oral drug delivery device, but the rotation axis can remain in the same position with respect to the housing.

In one or more example oral drug delivery devices, the first rotation axis is arranged at a center of the housing and/or the drug delivery device. In one or more example oral drug delivery devices, the first rotation axis is arranged at a longitudinal center of the housing. In one or more example oral drug delivery devices, the first rotation axis is arranged at a center of the housing as defined by an axis orthogonal to the longitudinal axis. The longitudinal axis can intersect the first rotation axis.

In one or more example oral drug delivery devices, the first rotation axis is arranged closer to an outer surface of the housing than a center of the housing. The first rotation axis can be spaced away from the longitudinal axis. For example, the first attachment part can be configured to rotate around an outer surface of the housing. In one or more example oral drug delivery devices, the second rotation axis is arranged closer to an outer surface of the housing than a center of the housing. The second rotation axis and the first rotation axis can be located on a same side of the housing, such as on a same side of the longitudinal axis. The second rotation axis and the first rotation axis can be located on a opposite sides of the housing, such as on opposite sides of the longitudinal axis.

In one or more example oral drug delivery devices, the first rotation axis is arranged closer to the first end than the second end. In one or more example oral drug delivery devices, the first rotation axis is arranged between the first end and a center along the longitudinal axis. In one or more example oral drug delivery devices, the first rotation axis is arranged closer to the first end than a center of the housing. For example, the housing can be defined as having a length L. The first rotation axis can be arranged at less than 0.4 L, such as less than 0.25 L from the first end of the housing. The first rotation axis can be arranged at 0.1 L, 0.2 L, 0.3 L, 0.4 L from the first end of the housing. A first rotation axis near the first end may allow for a longer first attachment part in turn increasing the chance of the first attachment part/distal end engaging with tissue during the rotation thereof outside the housing. The first rotation axis can be arranged at larger than 0.6 L, such as larger than 0.75 L from the second end of the housing.

As mentioned, the actuator is configured to rotate the first attachment part about the first rotation axis, such as in a first direction being parallel to or forming an angle less than 45 degrees with a distal direction being the direction in which the distal end of the first attachment part points parallel to the distal section. For example, the first attachment part can rotate with respect to the housing. In one or more example oral drug delivery devices, the first attachment part can rotate out of the first cavity. In one or more example oral drug delivery devices, the first attachment part, such as the distal end/distal section, can rotate (partially and/or fully) into the first cavity or a second cavity, e. g after being rotated out of the first cavity.

In one or more example oral drug delivery devices, the first attachment part is configured to rotate from an initial position to an attached position between 200 and 350 degrees. In other words, first attachment part may be rotated in the range from 200 degrees to 350 degrees to move from an initial position to an attached position. The first attachment part may be configured to rotate at least 100 degrees, such as at least 180 degrees or even at least 270 degrees, from the initial position to the attached position. In one or more example oral drug delivery devices, the first attachment part is configured to be in a non-attached position between 30 degrees and 350 degrees of the rotation of the first attachment part.

In one or more example oral drug delivery devices, in an initial position, the first attachment part, such as the distal end and/or the distal section of the first attachment part, is located within a first cavity of the housing. The first attachment part may, in an attached position, be located within the first cavity or a second cavity of the housing. In the attached position, the first attachment part, such as the distal end and/or the distal section of the first attachment part, can be at least partially located within the first cavity or within the second cavity. For example, the first distal end can be located within the second cavity in the attached position. In one or more example oral drug delivery devices, the first attachment part is located at and/or near the outer surface of the housing.

In one or more example oral drug delivery devices, the oral drug delivery device can contain a stopper. The stopper can be located inside of the second cavity. The stopper can be located outside of the housing. For example, a part of the outer surface of housing may form the stopper. The stopper is configured to stop rotation of the first attachment part. In one or more example drug delivery devices, the stopper is configured to rotate, e.g. about the first rotational axis or about a stopper rotational axis. The stopper rotational axis can be parallel to the first rotation axis. The stopper can be configured to rotate with the first attachment part. The stopper can be configured to rotate different from the first attachment part. The stopper can be a portion of the housing, such as the outer surface. For example, the stopper can be an indent in the housing. The stopper can be separate from the housing.

In one or more example oral drug delivery devices, the first attachment part is configured to deliver an active drug substance. The first attachment part, such as the first spike, may have one or more openings providing access to a (first) chamber in the housing and/or the first attachment part. In one or more example oral drug delivery devices, the (first) chamber is formed as or comprises a through-going bore in the first spike. The (first) chamber can be configured to hold, such as retain, an active drug substance. In one or more example oral drug delivery devices, the (first) chamber may be open from an inner volume of the housing and towards an outer part of the housing. In one or more examples, the (first) chamber may be inside the first spike so that when the first distal end of the first attachment part has penetrated the biological tissue, the active drug substance may be released from the (first) chamber and into the biological tissue via the first spike. This may e.g., be where the first spike is a tubular part, which has a first distal end in fluid communication with the (first) chamber.

In one or more example oral drug delivery devices, the oral drug delivery device only has a single attachment part (e.g., the first attachment part). In one or more example oral drug delivery devices, the oral drug deliver device comprises a second attachment part. The second attachment part may be connected on an opposite side of the housing from the first attachment part. The second attachment part may be connected on a same side of the housing as the first attachment part. The second attachment part can be rotational connected with the housing. The second attachment part can be fixed, e.g., non-rotation.

In one or more example oral drug delivery devices, the second attachment part is configured to rotate about the first rotation axis. For example, both the first attachment part and the second attachment part can be configured to rotate about the first rotation axis. In one or more example oral drug delivery devices, the second attachment part and the first attachment part are configured to rotate about the first rotation axis at the same time.

In one or more example oral drug delivery devices, the oral drug delivery device can comprise a second attachment part. The second attachment part can have a second distal end and a second proximal end. The second attachment part can be connected to and/or within the housing. The actuator can be engaged with the second attachment part and configured to rotate the second attachment part about a second rotation axis with respect to the housing. The second rotation axis can be different from the first rotation axis. The second rotation axis can be the same as the first rotation axis. In other words, the first attachment part and the second attachment part may share the same first hinge part.

In one or more example oral drug delivery devices, the second attachment part is configured to deliver an active drug substance. The second attachment part, such as the second spike, may have one or more openings providing access to the first chamber and/or a second chamber in the housing and/or the first attachment part. In one or more example oral drug delivery devices, the second chamber is formed as or comprises a through-going bore in the second spike. The second chamber can be configured to hold, such as retain, an active drug substance. In one or more example oral drug delivery devices, the second chamber may be open from an inner volume of the housing and towards an outer part of the housing. In one or more examples, the second chamber may be inside the second spike so that when the second distal end of the second attachment part has penetrated the biological tissue, the active drug substance may be released from the chamber and into the biological tissue via the second spike. This may e.g., be where the second spike is a tubular part, which has a second distal end in fluid communication with the second chamber.

In one or more example oral drug delivery devices, the actuator is configured to rotate the first attachment part and the second attachment part. In one or more example oral drug delivery devices, the actuator is configured to rotate the first attachment part and a second actuator is configured to rotate the second attachment part.

In one or more example oral drug delivery devices, the first attachment part is operably coupled with the second attachment part, and wherein rotation of the first attachment part rotates the second attachment part. For example, the first attachment part can be operably coupled with the second attachment part via one or more gears. Other connecting and/or intermediatory components can be used to operable couple the first attachment part with the second attachment part.

In one or more example oral drug delivery devices, the first attachment part comprises a first arm and a first spike. The first spike can be arranged at or comprise the first distal end. In one or more example oral drug delivery devices, the second attachment part comprises a second arm and a second spike The second spike can be arranged at or comprise the second distal end. In one or more example oral drug delivery devices, the first spike and the second spike overlap within the housing. In one or more example oral drug delivery devices, the first spike and the second spike overlap within the housing when the first attachment part and the second attachment part are in their respective initial positions. The overlap can be a longitudinal overlap. The overlap can be a radial overlap.

In one or more example oral drug delivery devices, the first arm extends at least half a longitudinal length of the housing. In one or more example oral drug delivery devices, the second arm extends at least half the longitudinal length of the housing.

In one or more example oral drug delivery devices, upon activation of the actuator, the first arm and the second arm are configured to cross one another within the housing. For example, the first arm and the second arm can have a combined length greater than a length of the oral drug delivery device. In one or more example drug delivery devices, both the first arm and the second arm can be located on a plane orthogonal to the longitudinal axis at the same time. The plane orthogonal to the longitudinal axis can be at a longitudinal center of the oral drug delivery device. The plane orthogonal to the longitudinal axis can be offset from the longitudinal center of the oral drug delivery device.

In one or more example oral drug delivery devices, the first rotation axis and the second rotation axis are coaxial. In one or more example oral drug delivery devices, the first rotation axis is separated by a distance from the second rotation axis. For example, the first rotation axis can be spaced apart from the second rotation axis.

In one or more example oral drug delivery devices, the oral drug delivery device can include an actuator. The actuator is engaged with the first attachment part. The actuator is configured to rotate the first attachment part about the first rotation axis. For example, the actuator is connected (e.g., directly connected) to the first attachment part. The actuator can be operably coupled to the first attachment part. In one or more example oral drug delivery devices, the actuator is configured to rotate the first attachment part about the first rotation axis and not rotate any portion of the housing. In other words, the actuator can be configured to rotate the first attachment part with respect to the housing. The actuator can be configured to rotate the first attachment part from the initial position to the attached position. The actuator may be, or may include a resilient part. The actuator may be a spring. The actuator may be a rotational or linear spring. As the actuator can be directly engaged with the first attachment part, there is not a need to unfold the first attachment part and then perform an additional action to attach the first attachment part to tissue. The first attachment part can attach to tissue in a single movement, namely the rotation around the first rotation axis.

The actuator, such as the resilient part, may be utilized to store energy, such as rotational energy or rotational force which can be applied to first attachment part, where the energy is stored in the actuator, such as the resilient part. Furthermore, when the energy is released, the force may be released to the first attachment part. The actuator, such as the resilient part, may e.g., be in the form of a helical spiral spring (mainspring) and/or a spiral torsion spring. The actuator can store energy in the mainspring by twisting the spiral tighter. The stored force of the mainspring may then rotate the attachment part in the opposing direction as the mainspring unwinds. Thus, the force of the mainspring may cause the first attachment part, and optionally the second attachment part, to travel in opposing directions, and where the attachment part(s) may pinch the biological tissue and either pinch the tissue or penetrate the tissue in order to attach the oral drug delivery device to the biological tissue.

In one or more example oral drug delivery devices, the actuator can be configured to move the first attachment part in relation to the second attachment part, such as configured to move the first distal end towards the second distal end. In one or more example oral drug delivery devices, the actuator can be configured to move the first distal end away from the second distal end on a first side of the housing, and then move the first distal end towards the second distal end on the second side of the housing. For example, the actuator mechanism is configured to translate the first attachment part in the first direction about the first rotation axis and the second attachment part in the second direction about the second rotation axis.

In one or more example oral drug delivery devices, the actuator is configured to move the first distal end towards the second distal end, such as from the initial position to the attached position. To move the first distal end towards the second distal end may be understood as reducing a distance between the first distal end and the second distal end. To move the first distal end towards the second distal end may be understood as reducing an angle between the first attachment axis and the second attachment axis, such as reducing an angle between the first attachment part and the second attachment part. In one or more example drug delivery devices, the actuator reduces the distance between the first distal end and the second distal end during rotation of the first body part with respect to the second body part around the central axis. In certain example embodiments, the drug delivery device may be in the attachment configuration when the first distal end is closest to the second distal end.

Thus, the attachment part(s) may be adapted to come into contact and/or penetrate tissue of the gastrointestinal tract. The rotation of the attachment part(s) may translate the attachment part(s) in such a way that they are capable of e.g., penetrating or pinching the mucous membrane in order to fix the oral drug delivery device at a location in the gastrointestinal tract, such as the stomach and/or gastrointestinal tissues and/or intestines. When the first attachment part and/or the second attachment part penetrates or pinches the biological tissue, the respective penetration point(s) in the biological tissue may be utilized to deliver an active drug substance from the oral drug delivery device into the biological tissue, and where the active drug substance may be introduced into the biological tissue that is beyond the mucous membrane. Thereby, the active drug substance may enter the bloodstream more easily than if the active drug substance is released in the stomach or intestinal lumen, and the drug delivery may be more effective. An example of this is when the active drug substance is insulin, where insulin may degrade inside the gastrointestinal tract and is not capable of being absorbed from the gastrointestinal tract, but where a mucous membrane has been penetrated, and the insulin released through the penetrated gastrointestinal wall, the insulin will remain intact and reach the bloodstream of the user via the blood vessels in the intestinal layer beyond the mucous membrane (surface).

In one or more example oral drug delivery devices, the drug delivery device comprises a locking mechanism. The locking mechanism may be configured to prevent rotation of the first attachment part and/or the second attachment part with respect to the housing, e.g. in the initial configuration. The shell may operate as a locking mechanism. The locking mechanism can be configured to release, such as dissolve, allowing rotation. The locking mechanism may be configured to prevent actuation of the actuator. The locking mechanism may be, for example, a cover, shell, and/or a collar around an outer surface of the drug delivery device, such as around an outer surface of the housing. The locking mechanism may partially cover an outer surface of the drug delivery device, such as around the outer surface of the housing. The locking mechanism may fully cover an outer surface of the drug delivery device, such as around the outer surface of the housing. Other locking mechanisms can be used, such as a dissolvable plug, and the particular locking mechanism is not limiting. Upon dissolution of the locking mechanism, the actuator is configured to rotate the first attachment part about the first rotation axis.

In one or more example oral drug delivery devices, the locking mechanism may be a capsule, such as a dissolvable (e.g., degradable, biodegradable) capsule. The capsule can partially and/or fully surround the oral drug delivery device. The capsule can partially and/or fully surround the housing. The oral drug delivery device can include a capsule and a locking mechanism. Upon dissolution of the capsule, the actuator is optionally configured to rotate the first attachment part about the first rotation axis.

In the initial configuration, the first attachment part may be in the first initial position (e.g., initial position) and/or the second attachment part may be in the second initial position. For example, the first initial position may be where the first attachment part, such as the first distal end, is located within the first cavity. A similar position can be taken for the second attachment part. Further, the actuator may be configured to, but prevented from, rotating the first attachment part and/or the second attachment part with respect to the housing. This may be the most compact configuration of the drug delivery device.

The oral drug delivery device can then transition from the initial configuration to the attached configuration via rotation of the first attachment part from the initial position to the attached position. This transition may occur due to actuation of the actuator. For example, due to a locking mechanism releasing. The transition from the initial configuration to the attached configuration may include translating, such as rotating, the first attachment part about a first rotation axis in relation to the housing. In other words, the first attachment part can translate, such as rotate out of the first cavity/housing. The transition from the initial configuration to the attachment configuration may also include translating the second attachment part about a second rotation axis in relation to the housing. In other words, the second attachment part can translate out of the second cavity/housing. The attachment position can be configured for attaching the drug delivery device to a patient.

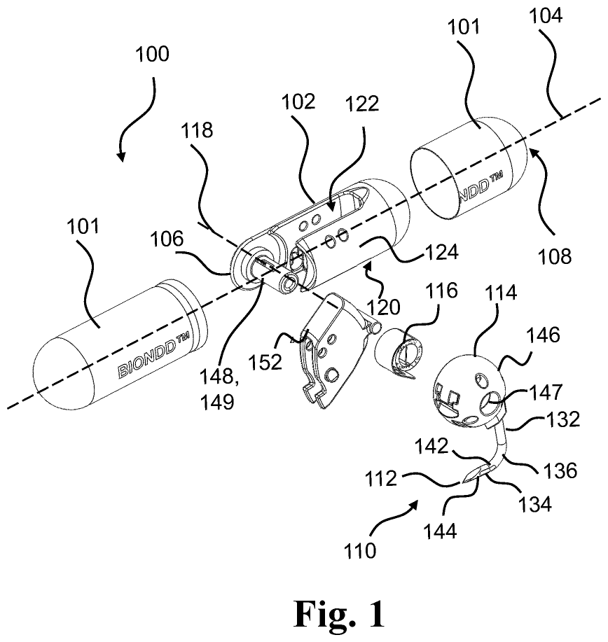

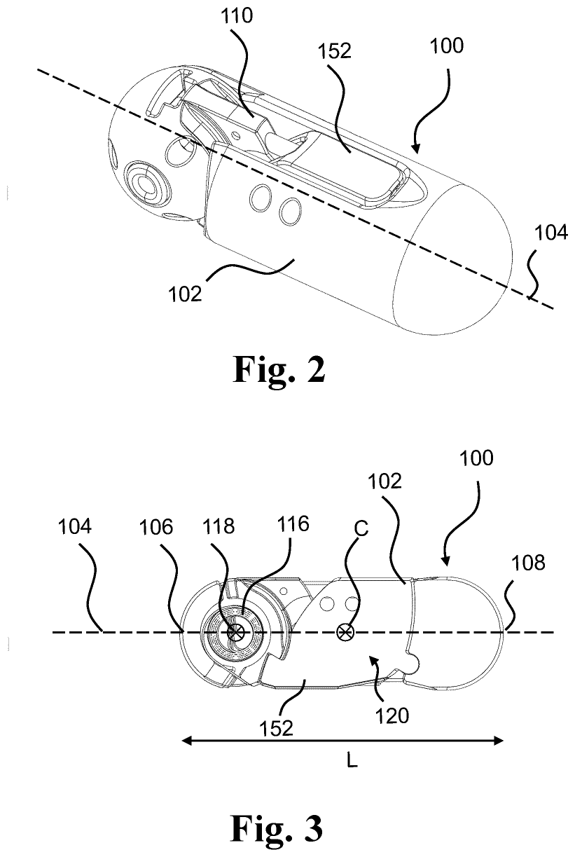

FIG. 1 shows an exploded view of an example oral drug delivery device according to the disclosure and FIGS. 2-6 show different views of the drug delivery device of FIG. 1. As shown, the oral drug delivery device 100 includes optional shell/capsule 101 (not shown in FIGS. 2-6) and a housing 102 accommodated in the shell 101. The housing 102 has a longitudinal axis 104 and extends between a first end 106 and a second end 108. Further, the oral drug delivery device 100 comprises a first attachment part 110. The first attachment part 110 has a first distal end 112 and a first proximal end 114. As shown, the first attachment part 110 is connected to and/or within the housing 102 and configured to deliver an active drug substance 154. The shell 101 operates as a locking mechanism and is configured to keep the first attachment part 110 in an initial position. Upon the shell 101 dissolving in the stomach/intestines, the first attachment part 110 is allowed to rotate to an attached position. The oral drug delivery device 100 further includes an actuator 116 engaged with the first attachment part 110. The actuator 116 (spiral blade spring) is configured to rotate the first attachment part 110 about a first rotation axis 118. The first rotation axis 118 forms an angle larger than 45 degrees with the longitudinal axis 104. In the illustrated example, the first rotation axis 118 is orthogonal to the longitudinal axis 104. In other implementations, the first rotation axis 118 forms an angle less than 15 degrees with the longitudinal axis 104.

The housing 102 forms a first cavity 120 and optionally a second cavity 122. The housing 102/first cavity 120 is configured to at least partially contain (e.g., surround, accommodate) components of the oral drug delivery device, such as at least part of the first attachment device 110.

In certain implementations, such as shown in FIG. 1, the first attachment part 110 includes a first arm 132, a first spike 134, and a bend 136 between the first arm 132 and the first spike 134. The first spike 134 is arranged at the first distal end 112, and the bend 136 has a bend in the range from 75 degrees to 105 degrees, such as a 95 degrees bend as shown, see also FIG. 5. The first spike 134 includes a proximal section 142 and a distal section 144, and wherein the distal section extends at an angle between 30-80 degrees or at an angle between 100-170 degrees from the longitudinal axis 104 in the attached position, see FIG. 5.

The first attachment part includes at the proximal end 114 a first attachment point embodied as first hinge part 146 comprising or forming a through-going bore 147 forming a hinge with a second hinge part 148 in the form of a rod 149 of the housing 102.

The oral drug delivery device 100 optionally comprises stopper 152 configured to stop the first attachment part 110 in the attached position, see FIG. 5.

FIGS. 2 and 3 show the first attachment part 110 in the initial position. FIG. 2 is a perspective view of the oral drug delivery device 100 without shell 101 and with the first attachment part 110 in the initial position, where the first attachment part 110 or at least parts 132, 134, 136 are positioned within the first cavity 120 of the housing 102.

FIG. 3 is a cross-sectional view of the oral drug delivery device 100. As clear from FIG. 3, the first rotation axis 118 is arranged closer to the first end 106 than the second end 108 of the housing, such as less than 0,25 L from the first end 106. For example, the first rotation axis 118 is arranged between the first end 106 and center C along the longitudinal axis 104. For example, the first rotation axis 118 is arranged closer to the first end 106 than center C of the housing 102. As shown in FIG. 3, in an initial position, the first attachment part 110 and the stopper 152 is located within first cavity 120 of the housing 102.

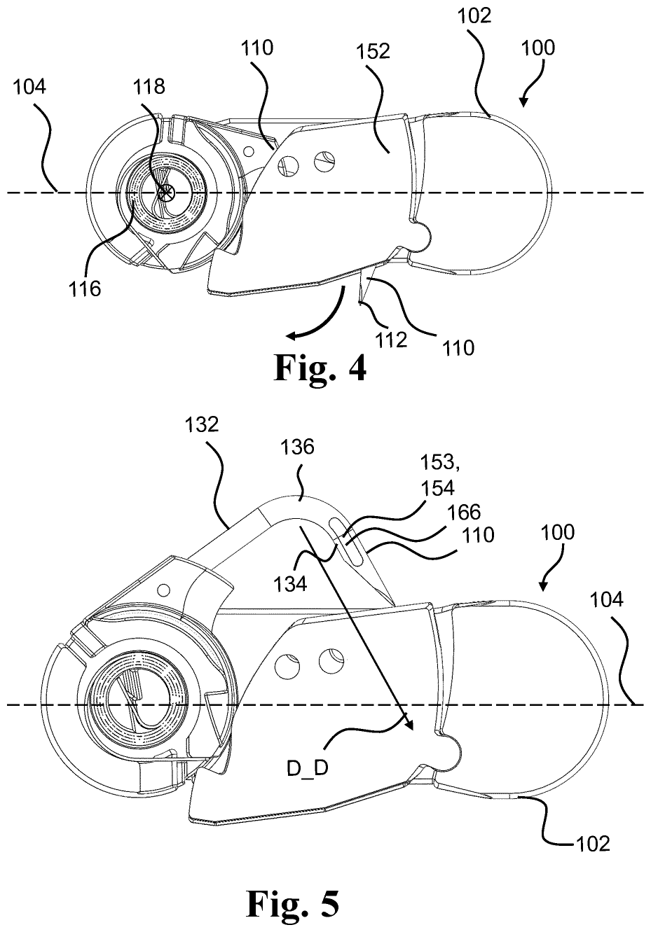

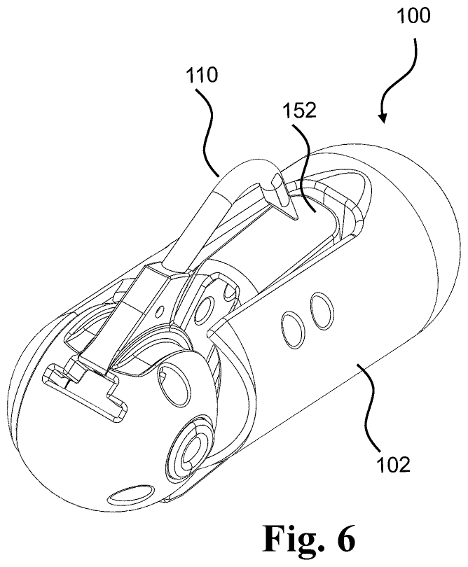

FIG. 4 is a cross-sectional view of the oral drug delivery device 100 where the first attachment device 110 has initiated rotation about the first rotation axis 118. The first attachment part 110 can rotate as shown by arrow in FIG. 4 to end in an attached position shown in FIGS. 5 and 6. The first attachment part 110 is configured to rotate from an initial position to an attached position between 200 and 350 degrees. The first attachment part 110 can stop in the attached position when the first distal end 112 abuts stopper 152.

FIG. 5 is a cross-sectional view of the oral drug delivery device 100 where the first attachment device 110 has rotated about the first rotation axis 118 to the attached position. The first attachment part 110, such as first spike 134, optionally comprises a chamber 153 holding an active drug substance 154. In one or more implementations, an active drug substance is embedded in the first attachment part 110.

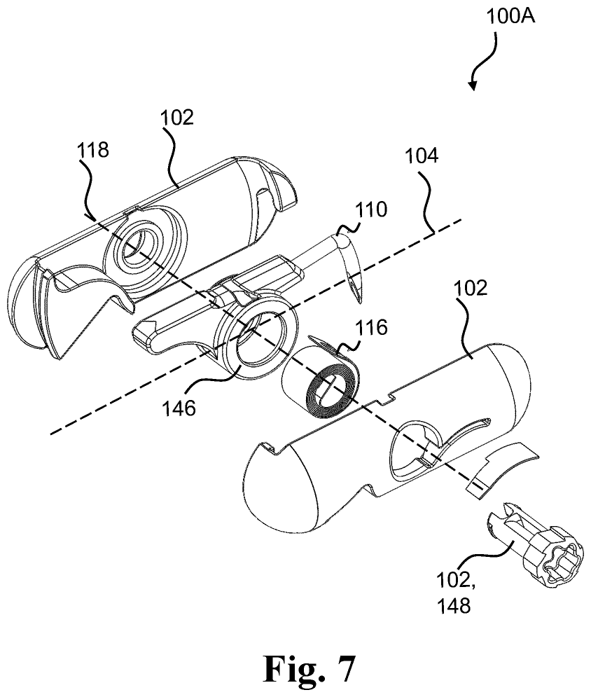

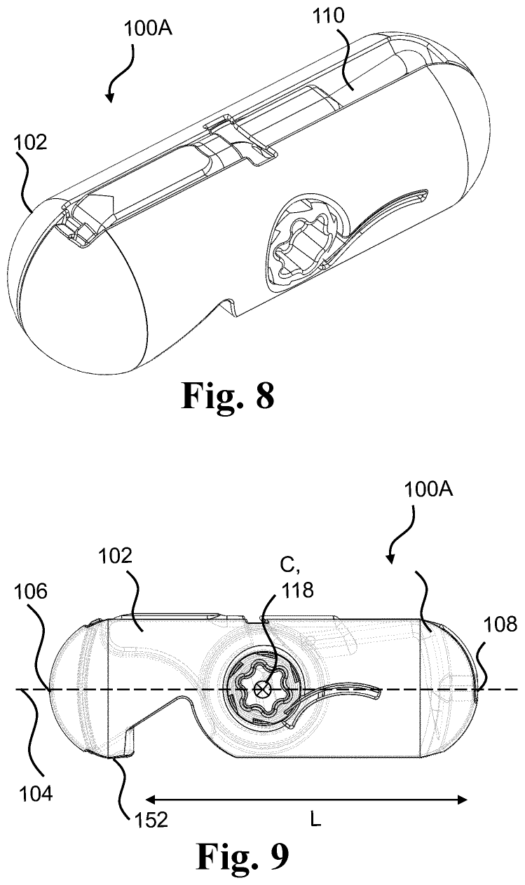

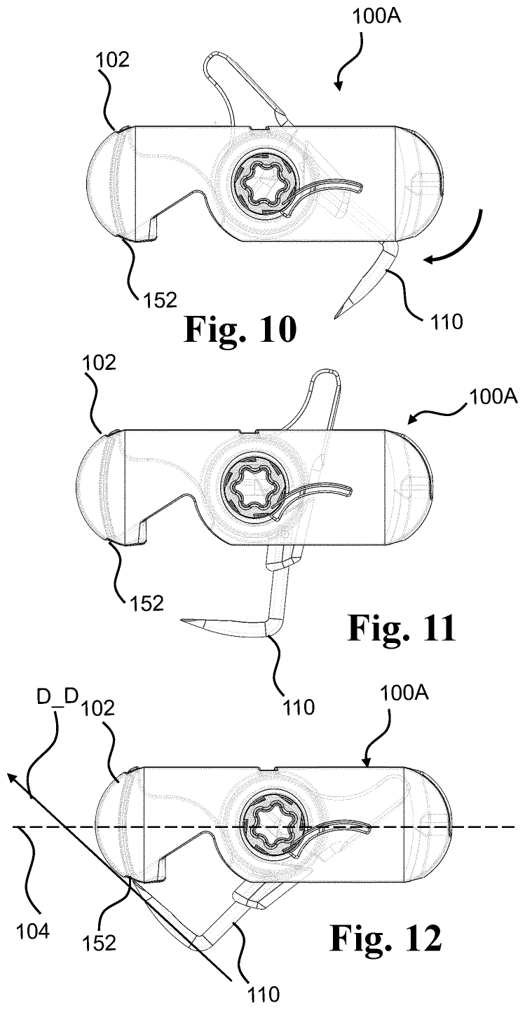

FIG. 7 shows an exploded view of an example oral drug delivery device 100A and FIGS. 8-12 show different views of the drug delivery device of FIG. 7. The drug delivery device 100A shown in FIGS. 7-12 can include any and/or all of the components of drug delivery device 100 shown in FIGS. 1-6 and will hereby use the same reference numbers. As shown, the first rotation axis 118 can be located at a center of the oral drug delivery device 100A, in particular at a center of the longitudinal axis 104. FIGS. 8-9 show the first attachment part 110 in the initial position, FIGS. 10-11 illustrate the first attachment part 110 rotating about the first rotation axis 118 as shownby arrow, and FIG. 12 shows the first attachment part 110 in the attached position, where the stopper 152 is an outer surface 124 of the housing 102.

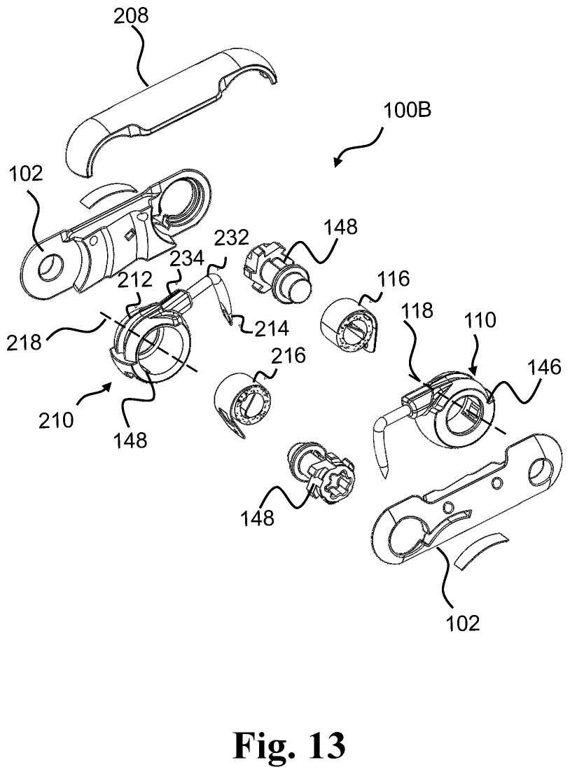

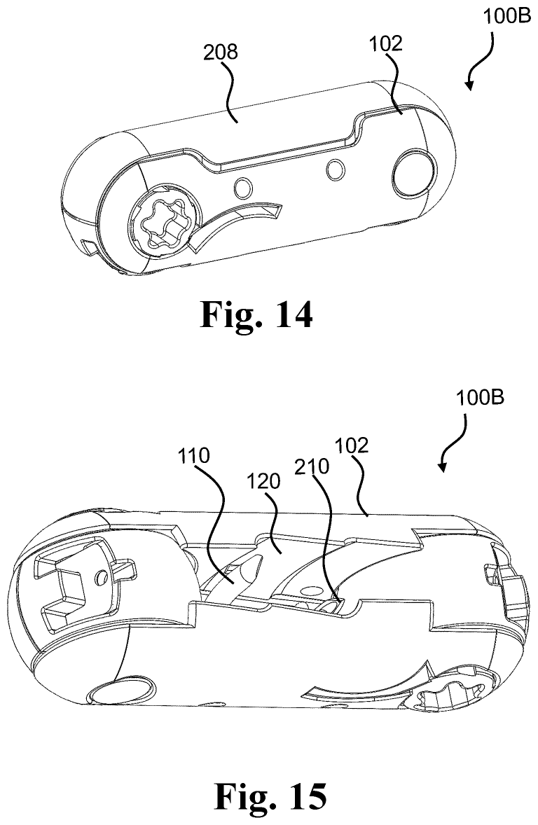

FIG. 13 shows an exploded view of an example oral drug delivery device 100B and FIGS. 14-20 show different views of the drug delivery device of FIG. 13. The drug delivery device 100B shown in FIGS. 13-20 can include any and/or all of the components of drug delivery device 100 shown in FIGS. 1-6 and/or drug delivery device 100A shown in FIGS. 7-12 and will hereby use the same reference numbers.

As shown, the oral drug delivery device 100B further includes a second attachment part 210. The second attachment part 210 has a second distal end 212 and a second proximal end 214. The second attachment part 210 connected within the housing 102. The actuator 116 is engaged with the second attachment part 210 and configured to rotate the second attachment part 210 about a second rotation axis 218 with respect to the housing 102. The first attachment part 110 is operably coupled with the second attachment part 210, and wherein rotation of the first attachment part 110 rotates the second attachment part 210. As shown in FIG. 13, the oral drug delivery device 100 can further include a second actuator 216 for rotation of the second attachment part 210.

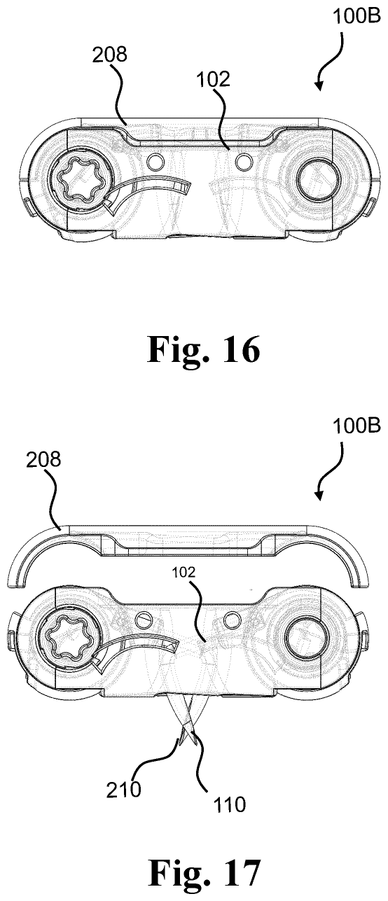

The first attachment part 110 includes a first arm 132 and a first spike 134, the first spike 134 arranged at the first distal end 112. The second attachment part 210 comprises a second arm 232 and a second spike 234, the second spike 232 arranged at the second distal end 212. As shown in FIG. 16, the first spike 134 and the second spike 234 overlap within the housing 102. Further, the first arm 132 extends at least half a longitudinal length of the housing 102 and the second arm 232 extends at least half the longitudinal length of the housing 102. Upon activation of the actuator 116, the first arm 132 and the second arm 132 are configured to cross one another within the housing 102, such as shown in FIG. 17.

Further, the first rotation axis 118 is separated by a distance from the second rotation axis 218.

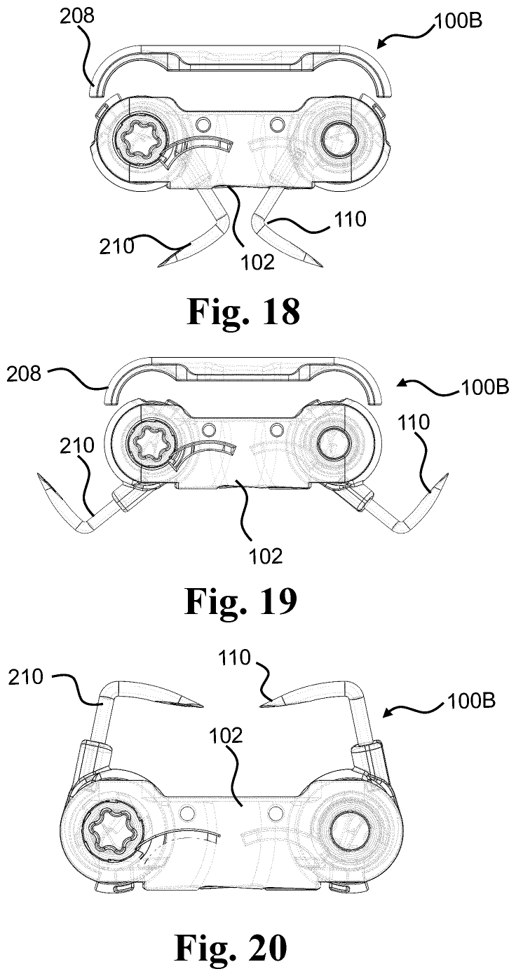

FIGS. 14-16 illustrate both attachment parts 110, 210 in respective initial positions. As shown, a locking mechanism 208, in this case a cover, prevents rotation of the attachment parts 110, 210. FIG. 17 illustrates the locking mechanism 208 releasing from the housing 102, thus allowing rotation of the attachment parts 110, 210, followed by FIGS. 18-19. FIG. 20 shows the attachment parts 110, 210 in the attached position.

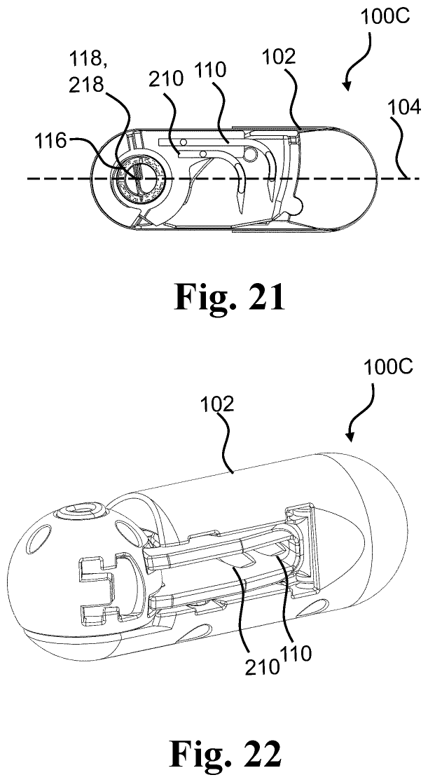

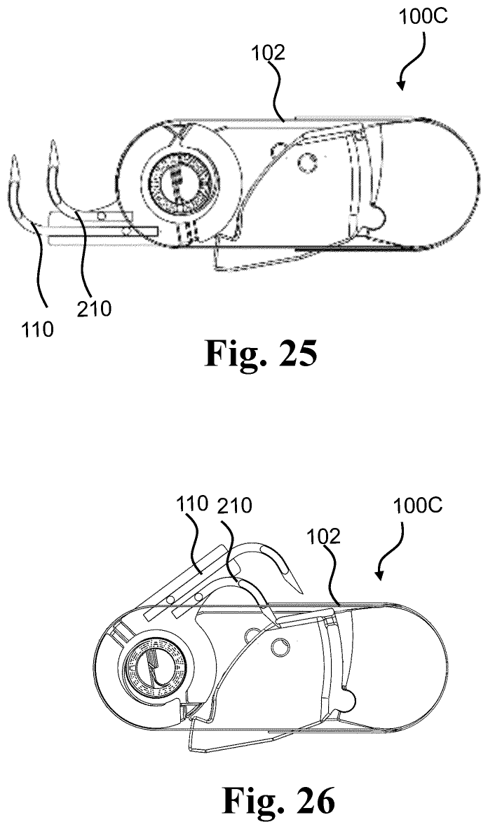

FIGS. 21-26 show perspective views of an example oral drug delivery device 100C. The drug delivery device 100C can include any and/or all of the components of drug delivery devices 100, 100A, 100B and will hereby use the same reference numbers.

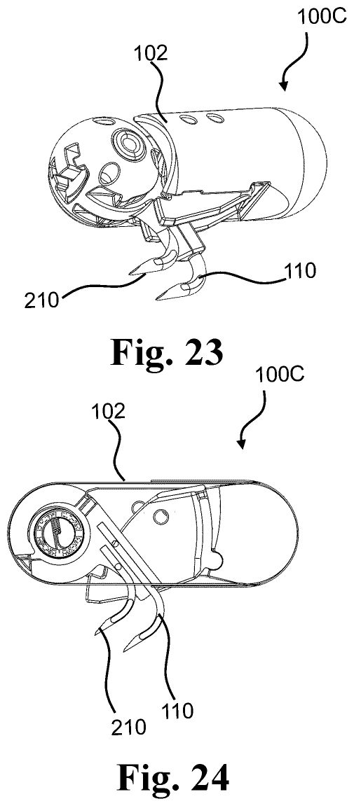

As shown in FIGS. 21-26, the first rotation axis 118 and the second rotation axis 218 are coaxial. This can occur because the first attachment part 110 and the second attachment part 210 can rotate in the same direction. As shown, the second attachment part 210 can be radially inward from the first attachment part 110. The actuator 116 can rotate both of the attachment parts 110, 210. FIGS. 21-22 show the attachment parts 110, 210 in the initial position. The actuator 116 can rotate the attachment parts 110, 210 through the positions shown in FIGS. 23-25 to end in the attached position of FIG. 26.

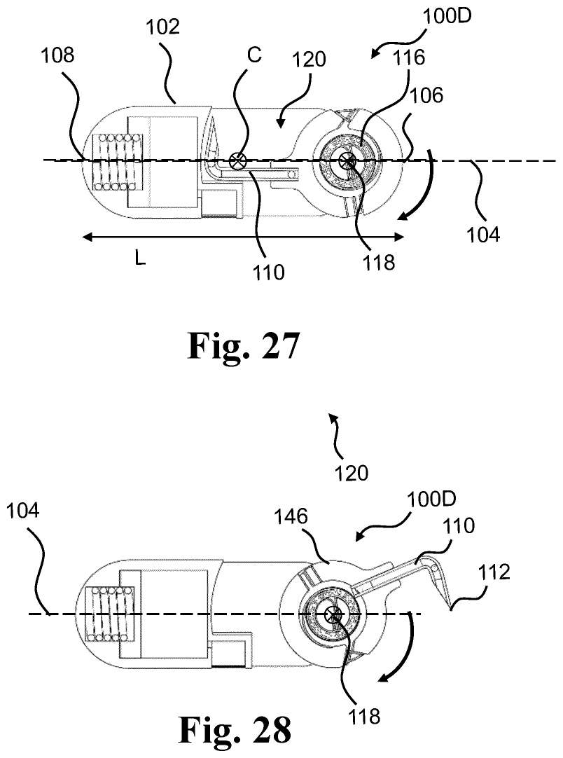

FIGS. 27-30 show perspective views of an example oral drug delivery device 100D. The drug delivery device 100D can include any and/or all of the components of drug delivery devices 100, 100A, 100B, 100C and will hereby use the same reference numbers.

FIG. 27 is a cross-sectional view of the oral drug delivery device 100D in initial configuration without shell 101 and with the first attachment part 110 in the initial position, where the first attachment part 110 or at least parts thereof are positioned within the first cavity 120 of the housing 102. As clear from FIG. 27, the first rotation axis 118 is arranged closer to the first end 106 than the second end 108 of the housing and even closer to the first end than the center C, such as less than 0,25 L from the first end 106.

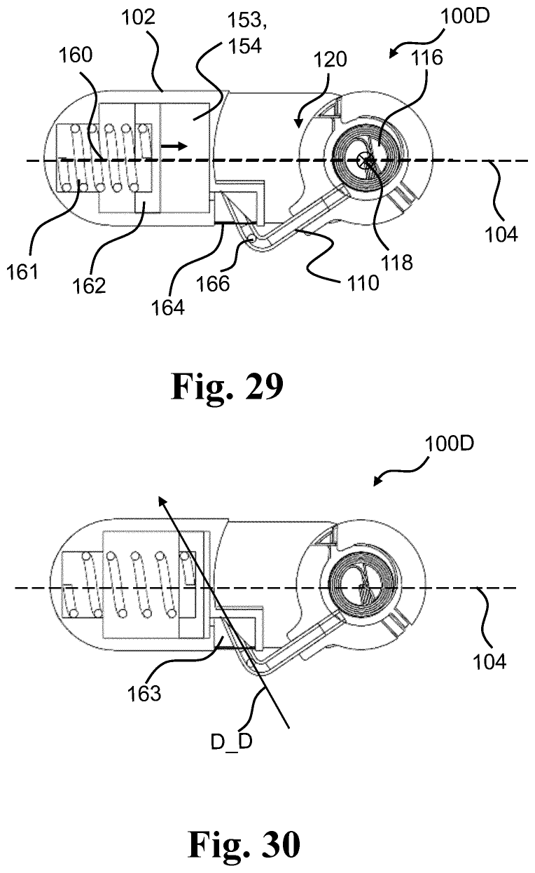

FIG. 28 is a cross-sectional view of the oral drug delivery device 100D where the first attachment device 110 has initiated rotation about the first rotation axis 118. The first attachment part 110 can rotate as shown by arrow in FIG. 28 to end in an attached position shown in FIGS. 29 and 30. The first attachment part 110 is configured to rotate from an initial position to an attached position between 200 and 350 degrees. The first attachment part 110 can stop in the attached position when the first distal end 112 abuts stopper 152.

FIGS. 29 and 30 are cross-sectional views of the oral drug delivery device 100D where the first attachment device 110 has rotated about the first rotation axis 118 to the attached position. The housing 102 comprises a chamber 153 holding an active drug substance 154. In the attached position, the first attachment part 110, such as first spike 134, activates injector 160 (spring 161 and piston 162) arranged in housing 102, e.g. by penetrating membrane 164 covering second chamber 163 to form a fluid connection from the chamber 153 to an opening 166 in hollow spike and/or hollow bend of the first attachment device 110 to allow active drug substance to flow from chamber 153 out of opening 166. FIG. 30 shows the oral drug delivery device 100D after the active drug substance.

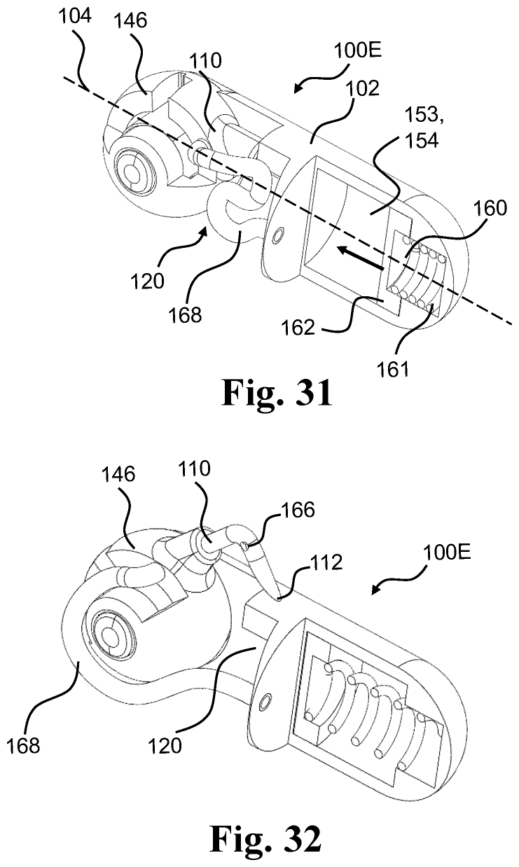

FIGS. 31-32 show perspective views of an example oral drug delivery device 100E. The drug delivery device 100E can include any and/or all of the components of drug delivery devices 100, 100A, 100B, 100C, 100D and will hereby use the same reference numbers.

FIG. 31 is a cross-sectional view of the oral drug delivery device 100E in initial configuration without shell 101 and with the first attachment part 110 in the initial position, where the first attachment part 110 or at least parts thereof are positioned within the first cavity 120 of the housing 102. The housing 102 comprises a chamber 153 holding an active drug substance 154. The chamber 153 is in fluid connection with the first attachment part 110 via tube 168. The oral drug delivery device 100E comprises an injector 160 (spring 161 and piston 162) arranged in the housing 102

FIG. 32 is a cross-sectional view of the oral drug delivery device 100E where the first attachment device 110 has rotated about the first rotation axis perpendicular to the longitudinal axis 104 to the attached position. In the attached position, the injector 160 (spring 161 and piston 162) has been activated to eject the active drug substance 154 from the chamber 153 to distal end 112 and/or opening 166 (optional) in hollow spike and/or hollow bend of the first attachment device 110 to allow active drug substance 154 to flow from chamber 153 via the tube 168 out of distal end 112 and/or opening 166. In one or more examples of oral drug delivery device 100E, the injector may be triggered or actuated by a dissolvable plug (not shown) in distal end 112/opening 166. Thereby correct sequential activation of the injector may be ensured.

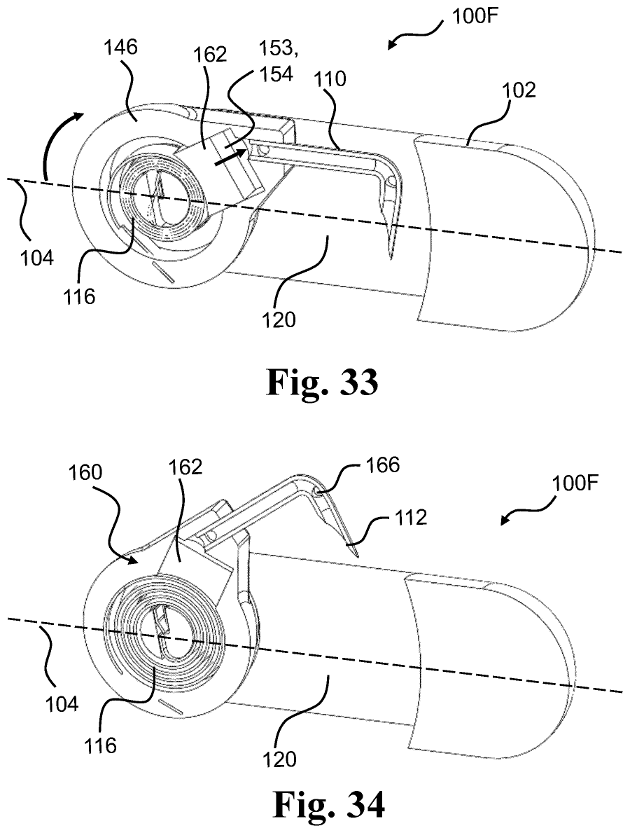

FIGS. 33-34 show perspective views of an example oral drug delivery device 100F. The drug delivery device 100E can include any and/or all of the components of drug delivery devices 100, 100A, 100B, 100C, 100D, 100E and will hereby use the same reference numbers.

FIG. 33 is a cross-sectional view of the oral drug delivery device 100F in initial configuration without shell 101 and with the first attachment part 110 in the initial position, where the first attachment part 110 or at least parts thereof are positioned within the first cavity 120 of the housing 102. The first attachment part 110 comprises a chamber 153 holding an active drug substance 154, the chamber 153 arranged in first hinge part 146. The oral drug delivery device 100E comprises an injector 160 (piston 162 activated/moved by actuator 116) arranged in the first hinge part 46 of the first attachment part 110.

FIG. 34 is a cross-sectional view of the oral drug delivery device 100F where the first attachment device 110 has rotated about the first rotation axis perpendicular to the longitudinal axis 104 to the attached position. In the attached position, the injector 160 (actuator 116 and piston 162) has been activated to eject the active drug substance 154 from the chamber 153 to distal end 112 and/or opening 166 (optional) in hollow spike and/or hollow bend of the first attachment device 110 to allow active drug substance 154 to flow from chamber 153 out of distal end 112 and/or opening 166. In one or more examples of oral drug delivery device 100F, the injector 160 may be triggered or actuated by a dissolvable plug (not shown) in distal end 112/opening 166. Thereby correct sequential activation of the injector may be ensured.

Also disclosed are oral drug delivery devices according to any of the following items.

Item 1. An oral drug delivery device comprising:
a housing having a longitudinal axis and extending from a first end to a second end;
a first attachment part having a first distal end and a first proximal end, the first attachment part connected to and/or within the housing and configured to deliver an active drug substance; and
an actuator engaged with the first attachment part and configured to rotate the first attachment part about a first rotation axis;
wherein the first rotation axis forms an angle larger than 45 degrees with the longitudinal axis.

Item 2. Oral drug delivery device of Item 1, wherein the first rotation axis is orthogonal to the longitudinal axis.

Item 3. Oral drug delivery device according to Item 1, wherein the first rotation axis forms an angle less than 15 degrees with the longitudinal axis.

Item 4. Oral drug delivery device of any of the preceding Items, wherein the first attachment part is configured to rotate from an initial position to an attached position between 200 and 350 degrees.

Item 5. Oral drug delivery device of any of the preceding Items, wherein in an initial position, the first attachment part is located within a first cavity of the housing and in an attached position the first attachment part is located within a second cavity of the housing.

Item 6. Oral drug delivery device of any of the previous Items, wherein the first rotation axis is stationary.

Item 7. Oral drug delivery device according to any one of the preceding Items, wherein the first rotation axis is arranged closer to an outer surface of the housing than a center of the housing.

Item 8. Oral drug delivery device according to any of the preceding Items, wherein the first rotation axis is arranged closer to the first end than the second end.

Item 9. Oral drug delivery device according to Item 8, wherein the first rotation axis is arranged between the first end and a center along the longitudinal axis.

Item 10. Oral drug delivery device according to Item 9, wherein the first rotation axis is arranged closer to the first end than a center of the housing.

Item 11. Oral drug delivery device according to any of the preceding Items, wherein the first attachment part comprises a first arm and a first spike, the first spike arranged at the first distal end, wherein the first spike includes a bend.

Item 12. Oral drug delivery device according to Item 11, wherein the first spike comprises a proximal section and a distal section, and wherein the distal section extends at an angle between 30-80 degrees or at an angle between 100-170 degrees from the longitudinal axis in an attached position.

Item 13. Oral drug delivery device according to any of the preceding Items, further comprising a second attachment part having a second distal end and a second proximal end, the second attachment part connected within the housing, wherein the actuator is engaged with the second attachment part and configured to rotate the second attachment part about a second rotation axis with respect to the housing.

Item 14. Oral drug delivery device according to Item 13, wherein the first attachment part is operably coupled with the second attachment part, and wherein rotation of the first attachment part rotates the second attachment part.

Item 15. Oral drug delivery device according to any one of Items 13-14, wherein:
the first attachment part comprises a first arm and a first spike, the first spike arranged at the first distal end; and
the second attachment part comprises a second arm and a second spike, the second spike arranged at the second distal end; and
the first spike and the second spike overlap within the housing.

Item 16. Oral drug delivery device according to Item 15,
wherein the first arm extends at least half a longitudinal length of the housing; and
wherein the second arm extends at least half the longitudinal length of the housing.

Item 17. Oral drug delivery device according to any one of Items 15-16, wherein upon activation of the actuator, the first arm and the second arm are configured to cross one another within the housing.

Item 18. Oral drug delivery device according to any one of Items 13-17, wherein the first rotation axis and the second rotation axis are coaxial.

Item 19. Oral drug delivery device according to Items 13-17, wherein the first rotation axis is separated by a distance from the second rotation axis.

The use of the terms “first”, “second”, “third” and “fourth”, “primary”, “secondary”, “tertiary” etc. does not imply any particular order, but are included to identify individual elements.

Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the example embodiments may be implemented at least in part by means of both hardware and software, and that several “means”, “units” or “devices” may be represented by the same item of hardware.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt. /vol. % of, within less than or equal to 5 wt. /vol. % of, within less than or equal to 1 wt. /vol. % of, within less than or equal to 0.1 wt. /vol. % of, and within less than or equal to 0.01 wt. /vol. % of the stated amount.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

100, 100A, 100B, 100C, 100D, 100E, 100F oral drug delivery device
102 housing
104 longitudinal axis
106 first end
108 second end
110 first attachment part
112 first distal end
114 first proximal end
116 actuator
118 first rotation axis
120 first cavity
122 second cavity
124 outer surface
132 first arm
134 first spike
136 bend
142 proximal section
144 distal section
146 first hinge part
147 through-going bore
148 second hinge part
149 rod
152 stopper
153 chamber
154 active drug substance
160 injector
161 spring
162 piston
163 second chamber
164 membrane
166 opening
168 tube
208 locking mechanism
210 second attachment part
212 second distal end
214 second proximal end
216 second actuator
218 second rotation axis
232 second arm
234 second spike
C center of housing
L length of housing
D_D distal direction

1. An oral drug delivery device comprising:
a housing having a longitudinal axis and extending from a first end to a second end;
a first attachment part having a first distal end and a first proximal end, wherein the first attachment part is connected to and/or is within the housing and configured to deliver an active drug substance; and
an actuator engaged with the first attachment part and being configured to rotate the first attachment part about a first rotation axis,
wherein the first rotation axis forms an angle larger than 45 degrees with the longitudinal axis.

2-11. (canceled)

12. The oral drug delivery device of claim 1, wherein the first rotation axis is orthogonal to the longitudinal axis.

13. The oral drug delivery device according to claim 1, wherein the first attachment part is configured to rotate from an initial position to an attached position between 200 and 350 degrees.

14. The oral drug delivery device according to claim 1, wherein in an initial position, the first attachment part is located within a first cavity of the housing and in an attached position the first attachment part is located within a second cavity of the housing.

15. The oral drug delivery device according to claim 1, wherein the first rotation axis is stationary.

16. The oral drug delivery device according to claim 1, wherein the first rotation axis is arranged closer to an outer surface of the housing than a center of the housing.

17. The oral drug delivery device according to claim 1, wherein the first rotation axis is arranged closer to the first end than the second end.

18. The oral drug delivery device according to claim 17, wherein the first rotation axis is arranged between the first end and a center of the oral drug delivery device along the longitudinal axis.

19. The oral drug delivery device according to claim 18, wherein the first rotation axis is arranged closer to the first end than to the center of the oral drug delivery device.

20. The oral drug delivery device according to claim 1, wherein the first attachment part comprises a first arm and a first spike, the first spike arranged at the first distal end, wherein the first attachment part includes a bend.

21. The oral drug delivery device according to claim 20, wherein the first spike comprises a proximal section and a distal section, and wherein the distal section extends at an angle between 30-80 degrees or at an angle between 100-170 degrees from the longitudinal axis in an attached position.

22. The oral drug delivery device according to claim 1, wherein the first attachment part is configured to rotate at least 100 degrees from an initial position to an attached position.

23. The oral drug delivery device according to claim 22, wherein the first distal end is configured to exit the housing when transitioning from the initial position to the attached position.

24. The oral drug delivery device according to claim 1, wherein the first rotation axis is arranged at a longitudinal center of the housing.

25. The oral drug delivery device according to claim 1, wherein the oral drug delivery device contains a stopper configured to stop rotation of the first attachment part and formed in an outer surface of the housing.

26. The oral drug delivery device according to claim 1, wherein the oral drug delivery device comprises a shell, wherein the shell is configured to operate as a locking mechanism configured to prevent rotation of the first attachment part with respect to the housing.

27. The oral drug delivery device according to claim 1, wherein the first attachment part comprises a first spike, a first tip of the first spike forming the first distal end, wherein the first spike is curved.
